(19) United States
(12) Patent Application Publication
(10) Pub. No.: US 2010/0217297 A1
(43) Pub. Date: Aug. 26, 2010

(54) Surgical Knife Providing Enhanced Blade Visualization

(76) Inventor: Anita Nevyas-Wallace, Narberth, PA (US)

Correspondence Address: Saul Ewing LLP (Philadelphia);Attn: Patent Docket Clerk, Penn National Insurance Plaza, 2 North Second St., 7th Floor, Harrisburg, PA, 17101, US

(21) Appl. No.: 12/714,081

(22) Filed: Feb. 26, 2010

Related U.S. Application Data
(60) Provisional application No. 61/155,812, filed on Feb. 26, 2009.

(51) Int. Cl. A61F 9/007; A61B 17/32

(52) U.S. Cl. 606/166; 606/172

(57) ABSTRACT

A surgical knife providing enhanced visualization includes an elongated handle, a footplate and a non-transparent blade. The footplate is supported on the handle and has a foot positioned to ride along tissue of a surgical site. The non-transparent blade has a cutting edge and is supported on the handle with at least a portion of the cutting edge extending beyond the foot, to expose the cutting edge for making an incision. The non-transparent blade is visually observable with ease, particularly along a line of sight passing through translucent tissue adjacent an incision. The foot may be constructed of a transparent material to allow direct observation of the blade or the surgical site via a line of sight passing through the transparent foot. These features provide a knife well-suited to making precisely placed and precisely curved incisions of the type required for astigmatic keratotomy and limbal relaxation procedures.

### FIELD OF THE INVENTION

The present invention relates generally to a surgical knife providing enhanced visualization. More particularly, the present invention is directed to a novel surgical knife having a visually translucent or visually opaque blade and/or a visually transparent foot that enhance visualization of the blade and the surgical site during surgery, which is particularly useful when making arcuate keratotomy incisions, as in procedures involving astigmatic keratotomy or limbal relaxation incisions.

### DISCUSSION OF RELATED ART

Various surgical knives are known in the art for use in incisional keratotomy. Particularly well-known for radial keratotomy procedures is a particular knife that includes a diamond blade and a handle supporting a footplate having a pair of feet between which the blade protrudes. The handle includes an adjustment mechanism (e.g., micrometer) that adjusts the distance that a tip of the blade protrudes beyond the feet, for controlling the depth of cut of the knife. An improvement to this common knife includes a specially-configured blade having an enhancement portion. The enhancement portion includes a rearward blade edge that is sharpened only distally to provide an enhancement cutting edge, so that an unsharpened portion of the rearward blade edge can guide the blade through the incision as the enhancement portion finishes the cut by bringing it to full cut depth. Exemplary radial keratotomy knives having such enhancement portions are commercially-available as a Genesis™ knife, manufactured and/or sold by Accutome, Inc. of Malvern, Pa., and the similar DuoTrak™ knife described in U.S. Pat. Nos. 5,222,967 and 5,423,840, the entire disclosures of both of which are hereby incorporated herein by reference.

It has been observed that the feet of the footplates, which straddle the blade and have a guide surface designed to ride along the outer surface of the cornea to control the depth of cut of such knives, have been made of opaque materials, such as metal. Thus, it is impossible to visually observe a portion of the blade or surgical site that is positioned behind the opaque footplates relative to the surgeon's eye. It is believed that metals have been selected for their rigidity and ease of sterilization, without regard to their opacity.

Further, it has been observed that the blades of such knives have been made of transparent materials, such as diamonds. Thus, it is difficult to visually discern with precision the boundaries of the transparent blade and their positions relative to the adjacent tissues, which are typically translucent or transparent. It is believed that diamonds have been selected for their hardness and suitability for maintenance of a sharp cutting edge, without regard to their transparency.

FIG. 1 shows the anatomy of a human eye generally designated by reference numeral 10. The outer surface of the eye 10 is formed by a cornea 12 that terminates at the corneal margin or limbus 14 in the vicinity of an anatomical protuberance on the inner surface of the cornea known as the scleral spur. The ciliary muscle joins the iris. The ciliary muscle is connected by the zonular fibers to the capsule of the crystalline lens. Contraction of the ciliary muscle changes the shape and position of the lens, which results in focusing of the vision of the subject. The iris surrounds the pupil, where light passes through the lens onto the retina (not shown) for transmission of an image to the optic nerve and the brain.

It has been found that myopia can be surgically corrected by a radial keratotomy (RK) procedure. During the RK procedure, the surgeon uses a knife to make several relatively short incisions of a controlled depth in the cornea to produce a flattening effect on the cornea. As is apparent from the procedure's name, the incisions are made (or are intended to be made) radially, in a straight line, i.e., linearly. These incisions typically extend from the edge of the optical zone, illustrated by dotted lines 16, to within 1.5 millimeters of the limbus. Exemplary radial incisions designated by reference numeral 20 are shown in the outer surface of the cornea 12 of FIG. 2, which have the effect of flattening the cornea.

A typical surgical procedure involves marking the eye prior to making the incision, e.g. by making an impression with a marking ring, by making scratches on the surface of the eye, or by making markings with a dye pen. No matter the method, it is relatively easy for the surgeon to visualize, adjacent the leading and trailing sides of the blade, the marked starting and stopping points for the required incision, and to observe visually the straightness of the incision by observing the incision trailing the blade and the marked stopping point ahead of the blade, over a very short distance, typically about 2.5 mm.

### SUMMARY

A surgical knife providing enhanced visualization includes an elongated handle, a footplate supported on the handle, and a blade. In one embodiment, the blade is constructed from a non-transparent material. The footplate has at least one foot having a guide surface positioned to ride along tissue of a surgical site. In one embodiment, the foot is constructed from a transparent material. The blade has a cutting edge, and the blade is supported on the handle with at least a portion of the cutting edge extending beyond the guide surface of the foot.

Optionally, the knife includes an adjustment mechanism adjustable to vary the amount of the cutting edge extending beyond the guide surface of the foot.

A method for performing surgery using such a knife, comprises plunging a non-transparent blade into human tissue, visually observing a portion of the blade positioned within the human tissue by direct observation along a line of sight extending through tissue adjacent the blade and guiding the blade to make an incision in human tissue, while visually observing the portion of the blade through the tissue adjacent the blade. Optionally, the blade may be guided to make an arcuate incision selected from the group consisting of an astigmatic keratotomy incision and a limbal relaxing incision.

Another method for performing surgery using such a knife comprises plunging the blade into human tissue, visually observing a portion of the blade and/or the surgical site by direct observation along a line of sight extending through the transparent foot, and guiding the blade to make an incision in human tissue while visually observing the portion of the blade through the transparent foot.

### BRIEF DESCRIPTION OF THE FIGURES

An understanding of the following description will be facilitated by reference to the attached drawings, in which:

FIG. 1 is a diagrammatic view of a human eye showing exemplary incisions made in typical incisional keratotomy procedures;

FIG. 2 is a partial side plan view of a knife in accordance with an exemplary embodiment of the present invention;

FIG. 3 is a partial front plan view of the knife of FIG. 3; and

FIG. 4 is a perspective view showing the knife in use during eye surgery.

### DETAILED DESCRIPTION

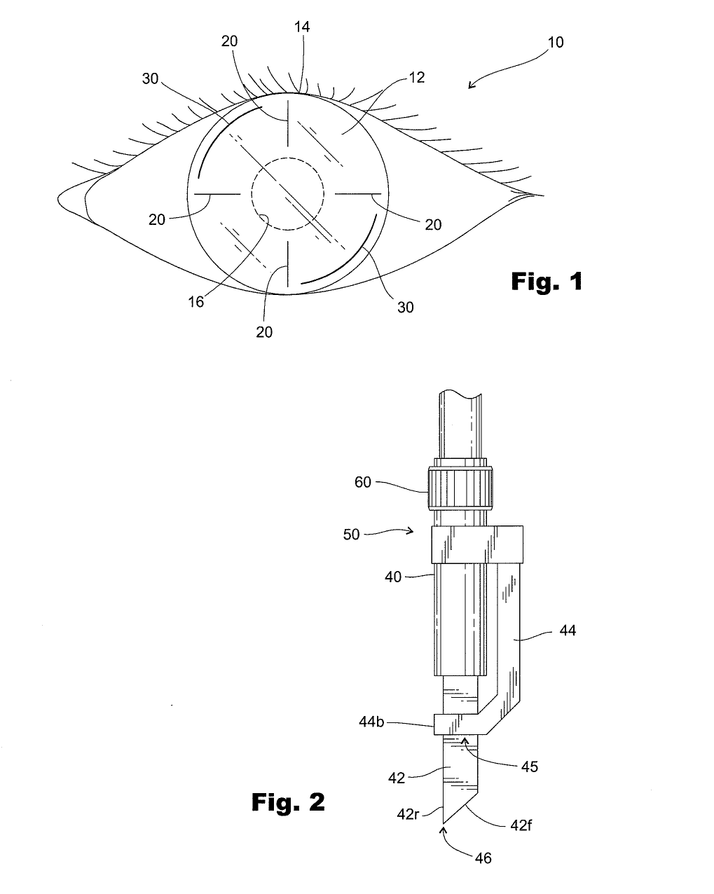

The present invention relates to a surgical knife that provides for enhanced visualization, and thus is particularly useful in procedures involving astigmatic keratotomy (AK) or limbal relaxation (LR) incisions. Such procedures, like radial keratotomy, require precise formation and placement of incisions. However, unlike radial keratotomy, the AK and LR procedures require formation of arcuate incisions. Further, each of these incisions is typically large relative to radial keratotomy incisions, often spanning approximately 30 degrees to 75 degrees of arc, and 3 mm to 6 mm in length. Exemplary AK and LR incisions are designated by reference numeral 30 in FIG. 1. Further, these incisions are intended to have a smooth curve and a substantially consistent radius, and/or to follow the natural contour of the cornea or limbus. Although it is relatively easy to adequately visually observe starting and stopping points for such arcuate incisions, it is exceedingly difficult to visually observe the incision and the structures of the eye to ensure that the incision is being formed with the appropriate curvature. Stated differently, it is relatively easy to visually observe that the incision is being made in a curved fashion, but it is relatively difficult to adequately visualize the extent and or consistency of the curvature, to maintain a constant radius, to follow the contour of the cornea/limbus, or to visually identify other reference points, so as to ensure that a proper incision is made, as such acts require visual observation of areas disposed laterally of the knife and the incision, which areas would be at least partially obscured by the opaque portions of the knife itself.

Accordingly, the present invention provides a surgical knife having a visually translucent or visually opaque blade and/or a visually transparent footplate. These features enhance visualization of the blade and the surgical site during surgery, and thus provide a surgical knife suitable for use in AK and LR procedures.

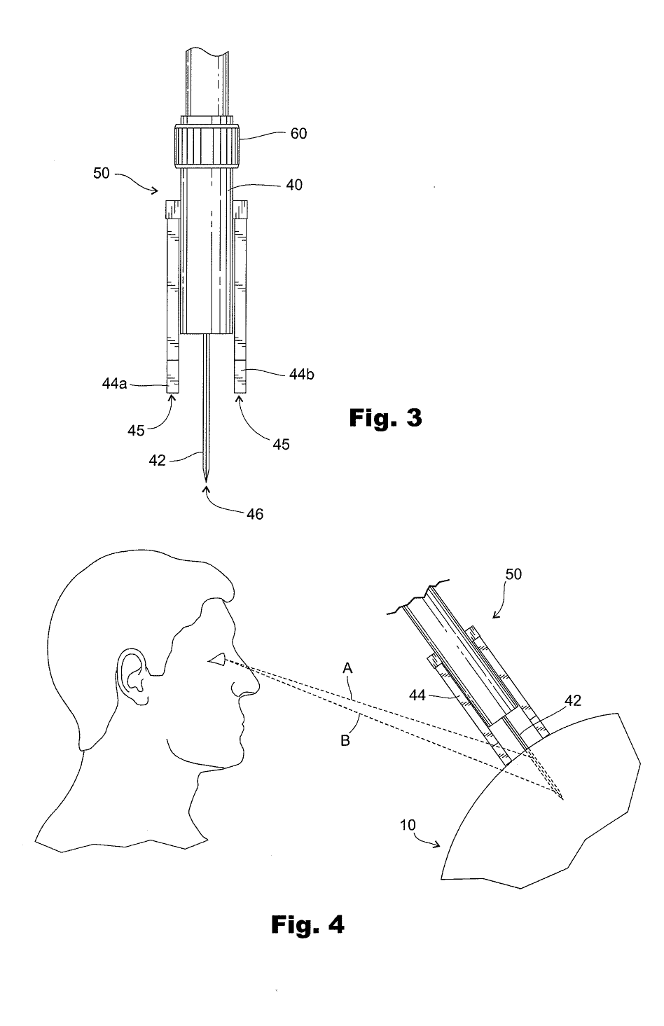

Referring now to FIGS. 2 and 3, a surgical knife 50 is shown in accordance with an exemplary embodiment of the present invention. The knife 50 includes a handle 40 supporting both a blade 42 and a footplate 44 having a pair of spaced feet 44a, 44b. The blade has at least one cutting edge 42a. The feet 44a, 44b are positioned to straddle the blade 42, and have a guide surface 45 designed to ride along the outer surface of the cornea 12 during the surgical procedure. In this exemplary embodiment, a shank of the blade 42 is received in a chuck of a micrometer supported on the handle 40. The micrometer is operable to control the depth of cut of the blade 42 by adjusting the distance that the tip 46 of the blade 42 projects beyond the guide surface of the footplate's feet 44a, 44b. Alternatively, other adjustment mechanisms may be included to permit adjustment of the amount of the blade's cutting edge that extends beyond the guide surface of the foot. The micrometer, and its mounting to the handle and blade are beyond the scope of the present invention, and such structures are well-known in the art for this purpose, and are thus not discussed in detail herein. An exemplary micrometer arrangement is disclosed in U.S. Pat. No. 5,674,233, the entire disclosure of which is hereby incorporated herein by reference. Another adjustment mechanism is disclosed in U.S. Pat. No. 4,569,133, the entire disclosure of which is hereby incorporated herein by reference. These exemplary adjustment mechanisms are operable to extend or retract the blade's tip relative to the guide surface 45 of the feet 44a, 44b by turning an adjustment knob 60.

In accordance with the present invention, a surgical knife 50 is provided with a non-transparent blade 42 constructed from a visually translucent or a visually opaque material. The blade may be part of the incisional keratotomy knife described herein, or may be incorporated into other knives having otherwise conventional structure.

A visually translucent material permits light to pass therethrough but diffuses it so that the translucent material is visually perceptible against a background. Examples of visually translucent materials include gemstones such as sapphires and naturally-occurring, synthetic, and colored diamonds. Any translucent material suitable for a surgical blade may be used. Particularly suitable for this purpose are black, or “carbonado” diamonds, which contain numerous dark inclusions that give them their dark appearance. It should be noted that suitable colored diamonds can be produced synthetically by ion implantation or other methods. It should be further noted that it is believed that black diamonds have been used as cutting blades for various surgical instruments, and that it is further believed that such black diamonds were selected for their hardness and for their less expensive cost, relative to transparent diamonds, and not for their visual properties.

A visually opaque material is not transparent or translucent, i.e., it does not permit light to pass therethrough. Any opaque material suitable for a surgical blade may be used. Examples of visually opaque materials include metals, such as stainless steel, titanium and aluminum, and ceramic.

Accordingly, a visually translucent or visually opaque blade may be visually observed easily, both before making an incision, and during making of an incision. Further, such a blade may be visually observed by the surgeon by direct observation of the blade while it is positioned within an incision in the ocular tissue, and more particularly, by direct observation of the blade along a line of sight that extends through transparent or translucent ocular tissue adjacent the incision.

An exemplary method for performing surgery, such as incisional keratotomy surgery, comprises plunging a visually translucent or visually opaque blade into human tissue, such as ocular tissue, visually observing a portion of the blade positioned within the human tissue, e.g., by direct observation along a line of sight (see exemplary line of sight B, FIG. 4) extending through transparent or translucent tissue adjacent the blade, and guiding the blade, while visually observing the portion of the blade positioned within the human tissue, to make an incision in the human tissue. For example, this method may be used to make arcuate astigmatic keratotomy incisions and/or limbal relaxing incisions.

In accordance with the present invention, the incisional keratotomy knife described herein, may be provided not only with a blade made from a visually translucent or a visually opaque material, but also with a pair of feet constructed from a visually transparent material.

A visually transparent material permits light to pass therethrough so that bodies situated beyond or behind it can be seen distinctly. Examples of visually transparent materials include glass and plastic, such as polycarbonate or poly[methyl-methacrylate], so that an object constructed from the materials appears visually clear. Any visually transparent material may be used, provided that the material is sufficiently rigid to act as a foot as described herein. Preferably, the transparent material is selected to have a refractive index similar to that of corneal tissue or of air.

The exemplary incisional keratotomy knife of FIGS. 2 and 3 is provided with visually transparent feet 44a, 44b and a visually translucent or visually opaque blade 42. Accordingly, this knife 50 has a blade 42 that may be visually observed easily, both before making an incision, and during making of an incision. Further, such a blade 42 may be visually observed by the surgeon by direct observation of the blade 42 while it is positioned within an incision in the ocular tissue. More particularly, the blade 42 may be visually observed by direct observation of the blade 42 through transparent or translucent ocular tissue adjacent the incision, and/or through a transparent feet 44a, 44b of the knife 50. Accordingly, such a knife 50 is particularly well-suited to making carefully-guided arcuate incisions for astigmatic keratotomy and limbal relaxation incisions.

An exemplary method for performing incisional keratotomy surgery comprises plunging a visually translucent or visually opaque blade into human tissue, such as ocular tissue, visually observing a portion of the blade, e.g., by direct observation along a line of sight (see exemplary line of sight A, FIG. 4) extending through a foot of the knife and/or through human tissue adjacent the blade, and guiding the blade, while visually observing the blade through the transparent foot, to make an incision in the human tissue having a controlled depth. The method may further involve withdrawing the blade from the human tissue when a visible stopping point marking, positioned on the ocular tissue, is reached by the blade, as observed by the surgeon through a transparent feet. For example, this method may be used to make arcuate astigmatic keratotomy incisions and/or limbal relaxing incisions.

Optionally, the blade 42 may be provided with an enhancement portion, e.g., to have at least one additional cutting edge, as described in U.S. Pat. No. 5,423,840, and the surgical methods described above may further include shaping the profile of the incision by moving a cutting edge of the blade that is shorter than the distance from the base of the incision to Bowman's membrane, along the incision at substantially the same controlled depth, as described therein.

While there have been described herein the principles of the invention, it is to be understood by those skilled in the art that this description is made only by way of example and not as a limitation to the scope of the invention.

What is claimed is:

1. A surgical knife comprising:
an elongated handle;
a footplate supported on the handle, said footplate comprising at least one foot having a guide surface positioned to ride along tissue of a surgical site; and
a non-transparent blade having a cutting edge, said blade being supported on said handle with at least a portion of said cutting edge extending beyond said guide surface of said foot.

2. The knife of claim 1, wherein said non-transparent blade is constructed from a translucent material.

3. The knife of claim 2, wherein said at least one foot is constructed from a transparent material.

4. The knife of claim 2, wherein said translucent material is selected from the group consisting of a sapphire, a naturally-occurring diamond, a synthetic diamond, a colored diamond, and a black diamond.

5. The knife of claim 1, wherein said non-transparent blade is constructed from a visually opaque material.

6. The knife of claim 5, wherein said at least one foot is constructed from a transparent material.

7. The knife of claim 5, wherein said visually opaque material selected from the group consisting of a metal or a ceramic.

8. The knife of claim 7, wherein said metal is selected from the group consisting of stainless steel, aluminum and titanium.

9. The knife of claim 1, wherein said at least one foot is constructed from a transparent material.

10. The knife of claim 1, wherein said footplate comprises a pair of feet and said blade is positioned to extend between said pair of feet, and wherein each of said pair of feet is constructed from a transparent material.

11. The knife of claim 9, wherein said transparent material has a refractive index substantially similar to that of corneal tissue.

12. The knife of claim 9, wherein said transparent material is selected from the group consisting of glass, polycarbonate and poly[methyl-methacrylate].

13. The knife of claim 1, further comprising an adjustment mechanism adjustable to vary the amount of the cutting edge extending beyond said guide surface of said foot.

14. The knife of claim 1, wherein said adjustment mechanism comprises a micrometer.

15. A surgical knife comprising:
an elongated handle;
a footplate supported on the handle, said footplate comprising at least one foot constructed from a transparent material, said foot having a guide surface positioned to ride along tissue of a surgical site; and
a blade having a cutting edge, said blade being supported on said handle with at least a portion of said cutting edge extending beyond said guide surface of said foot.

16. A method for performing surgery, comprising:
plunging a non-transparent blade into human tissue;
visually observing a portion of the blade positioned within the human tissue by direct observation along a line of sight extending through tissue adjacent the blade; and
guiding the blade to make an incision in human tissue while visually observing the portion of the blade through the tissue adjacent the blade.

17. The method of claim 16, wherein the plunging the non-transparent blade into human tissue comprises plunging a translucent or opaque blade into ocular tissue.

18. The method of claim 17, wherein guiding the blade to make an incision in human tissue comprises guiding the blade to make an arcuate incision selected from the group consisting of an astigmatic keratotomy incision and a limbal relaxing incision.

19. A method for performing surgery, comprising:
grasping a surgical knife comprising:
an elongated handle;
a footplate supported on the handle, the footplate comprising at least one foot having a guide surface positioned to ride along tissue of a surgical site, the at least one foot being constructed from a transparent material;
a blade having a cutting edge, the blade being supported on the handle with at least a portion of the cutting edge extending beyond the guide surface of the foot;
plunging the blade into human tissue;
visually observing a surgical site by direct observation along a line of sight extending through the transparent foot; and
guiding the blade to make an incision in human tissue while visually observing the portion of the blade through the transparent foot.

20. The method of claim 19, further comprising:
withdrawing the blade from the human tissue when the blade is visually observed to have reached a visible stopping point marking on the human tissue.

21. The method of claim 20, wherein the plunging the blade into human tissue comprises plunging a translucent or opaque blade into ocular tissue.

22. The method of claim 21, wherein guiding the blade to make an incision in human tissue comprises guiding the blade to make an arcuate incision selected from the group consisting of an astigmatic keratotomy incision and a limbal relaxing incision.
